(19) United States
(12) Patent Application Publication
(10) Pub. No.: US 2009/0226366 A1
(43) Pub. Date: Sep. 10, 2009

(54) Choline: Early Detector of Cancer

(75) Inventor: Fatma Jassab Faleh Al-Saeedi, Safat (KW)

Correspondence Address: FURR LAW FIRM, 2622 DEBOLT ROAD, UTICA, OH, 43080, US

(73) Assignee: UNIVERSITY OF KUWAIT, Safat (KW)

(21) Appl. No.: 12/045,020

(22) Filed: Mar. 9, 2008

(51) Int. Cl. A61K 51/00; C12Q 1/02; A61P 43/00

(52) U.S. Cl. 424/1.11; 435/29

(57) ABSTRACT

The current invention detects cancer in its early stages as proliferation measurement. This material “choline” can be tagged to several radioactive substances that can be used noninvasively to detect and monitor therapy, treatment and the management of cancer.

### FIELD OF THE INVENTION

The present invention relates to early detection of cancer and more particularly the use of choline in that detection.

### BACKGROUND OF THE INVENTION

There are have been many different methods and devices used to detect different types of cancers, but there exists a need for early ways to detect cancer.

### PRIOR ART REFERENCE AND DISCUSSION

U.S. Pat. No. 6,756,063 by Kiss and issued on Jun. 29, 2004 is for methods and compositions for the treatment of human and animal cancers. It discloses methods and compositions for altering the viability of cells, particularly cancers in animals and humans. The compositions of the invention are formed from a set of components comprising one or more of the following: a dithiocarbonyl, preferably dithiocarbamate, compound; a divalent metal ion; a modulator of cellular glutathione levels; and an inhibitor of the phosphorylation of choline. The compositions described herein induce a relatively selective and rapid effect on the viability of cancer cells by inducing a mixture of apoptotic and necrotic cell death, with the dominant pathway being apoptosis.

Particularly preferred active compositions comprise all four components, although combinations of fewer components can be fully effective in certain tumors.

### SUMMARY OF THE INVENTION

The current invention detects cancer in its early stages as proliferation measurement. This material “choline” can be tagged to several radioactive substances that can be used noninvasively to detect and monitor therapy, treatment and the management of cancer.

### BRIEF DESCRIPTION OF THE DRAWINGS

The above and other aspects, features, and advantages of the present invention will be better and more fully understood by those skilled in the art with reference to the following detailed and more particular description of specific and preferred embodiments thereof, presented in conjunction with the following drawings to show how the same may be carried into effect, wherein:

FIG. 1 is the chemical composition of Choline.

### DETAILED DESCRIPTION OF BEST MODE FOR CARRYING OUT THE INVENTION

There will now be described, by way of example only, the best mode contemplated by the inventor for carrying out the invention. In the following description numerous specific details are set forth in order to provide a thorough understanding of the present invention. It will be apparent, however, to one skilled in the art, that the present invention may be practiced without limitation to these specific details. In other instances, well known methods and structures have not been described in detail so as not to unnecessarily obscure the present invention.

The present invention relates to early detection of cancer and more particularly the use of choline in that detection. This invention helps in the treatment and the management of cancer.

It is well known that cancer is an uncontrolled increase in cell proliferation. With increase in cell proliferation, the need to synthesize cell membranes and structures is increased also. Choline is the precursor for building these cell membranes and structures and it is an essential component for cells structure and signaling.

The current invention allows the detection of cancer in its early stages as proliferation measurement. This material “choline” can be tagged to several radioactive substances that can be used noninvasively to detect and monitor therapy, treatment and the management of cancer.

Choline is an organic compound, classified as an essential nutrient and usually grouped within the Vitamin B complex. This natural amine is found in the lipids that make up cell membranes and in the neurotransmitter acetylcholine.

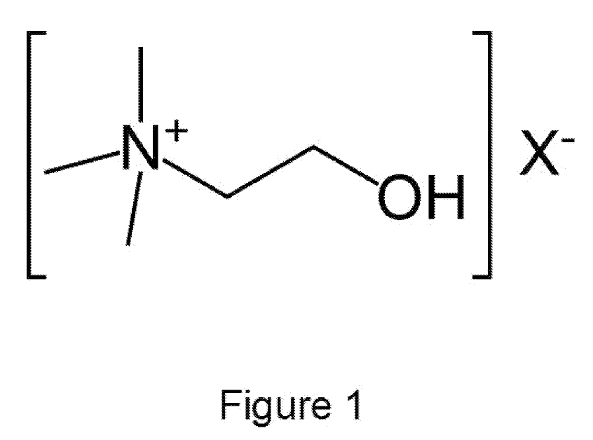

Choline is a quaternary saturated amine with the chemical formula: (CH₃)₃N⁺CH₂CH₂OHX⁻. where X⁻ is a counterion such as chloride (see choline chloride), hydroxide or tartrate as shown in FIG. 1.

Choline and its metabolites are needed for three main physiological purposes: structural integrity and signaling roles for cell membranes, cholinergic neurotransmission (acetylcholine synthesis), and as a major source for methyl groups via its metabolite, trimethylglycine (betaine) that participates in the S-adenosylmethionine synthesis pathways.

The medical profession relies heavily on direct radiation fields and radioactive isotopes for identifying and treating disease. In addition, radioactive materials are used extensively to test new drugs and conduct research into cures for disease.

To make radioactive materials, a cyclotron uses electric current to accelerate atomic particles, which strike the non-radioactive “target” material, turning it into a radioactive isotope—variant—of its original structure. For example, when the non-radioactive “target” element cobalt is struck by neutrons in a reactor, it is transformed into a radioisotope—cobalt-60—which is used to treat cancer and sterilize medical and consumer products. This is done to Choline in the current invention.

This material “choline,” that has been tagged with one or more radioactive substances, is ingested by the patient. The choline is then used by the body, such as being used in cell membranes, structural integrity and signaling roles for cell membranes, cholinergic neurotransmission (acetylcholine synthesis), and as a major source for methyl groups via its metabolite, trimethylglycine (betaine) that participates in the S-adenosylmethionine synthesis pathways which will allow its measurement as a detection for cancer.

### Equivalents

From the foregoing description, one skilled in the art can easily ascertain the essential characteristics of this invention and, without departing from the spirit and scope thereof, can make various changes and modifications of the invention to adapt it to various usages and conditions. Such variations and changes may include, for example, altering the number of components in the housing or using equivalents. It is believed that such can be accomplished without excessive experimentation. In any case, any such variations are all claimed under the scope of this invention.

The methods of the present invention have been explained with reference to plurality of references the teachings of which are all incorporated herein by reference.

This invention has been described hereinabove, although with reference to a plurality of illustrative and preferred embodiments, it is to be understood that is in no way to be construed as limiting but only to provide examples. However, it is readily appreciated that, from reading this disclosure, the invention may be embodied in other specific forms without departing from the spirit or essential characteristics or attributes to bring modifications by replacing some elements of this invention as practiced by their equivalents, which would achieve that same goal thereof and accordingly reference should be made to the appended claims, rather than to the foregoing specification, as indicating the scope of the invention. Accordingly, those skilled in the art will recognize or be able to ascertain, using no more than routine experimentation, many equivalents to the specific embodiments and the scope of the invention being indicated by the appended claims described herein. Such equivalents, obvious variations, and all changes which come within the meaning and equivalency of the claims are therefore intended to be encompassed therein and are deemed covered by the claims of this invention.

From the foregoing description, one skilled in the art can easily ascertain the essential characteristics of this invention and, without departing from the spirit and scope thereof, can make various changes and modifications of the invention to adapt it to various usages and conditions. Such variations and changes may include, for example, altering the number of components in the housing or using equivalents. It is believed that such can be accomplished without excessive experimentation. In any case, any such variations are all claimed under the scope of this invention.

We claim:

1. A process to detect cancer comprising:
using choline that has been tagged with a radioactive substances.

2. A process according to claim 1 further comprising:
having a patient ingest the choline.

3. A process according to claim 1 further comprising:
having said choline attached to cell membranes.

4. A process according to claim 3 further comprising:
measuring the amount and locations that choline has attached to cell membranes.

5. A process according to claim 1 further comprising:
having said choline absorded in the body.

6. A process according to claim 5 further comprising:
measuring the amount and locations that choline has been absorbed in the body.
